(19) United States
(12) Patent Application Publication
(10) Pub. No.: US 2007/0117879 A1
(43) Pub. Date: May 24, 2007

(54) Light-curing, self-etching, one-component dental adhesive containing nanoparticles

(75) Inventors: Marcus Hoffmann, Usingen (DE); Albert Erdrich, Bad Nauheim (DE)

Correspondence Address: Norris, McLaughlin & Marcus P.A., 18th Floor, 875 Third Avenue, New York, NY, 10022, US

(73) Assignee: Heraeus Holding GmbH, Hanau (DE)

(21) Appl. No.: 11/506,139

(22) Filed: Aug. 17, 2006

(30) Foreign Application Priority Data
Aug. 29, 2005 (DE) 10 2005 041 014.6

(51) Int. Cl. A61K 6/00

(52) U.S. Cl. 523/118

(57) ABSTRACT

Light-curing, self-etching one-component dental adhesives containing at least one acrylate or methacrylate monomer, at least one acidic component, at least one photoinitiator, at least one solvent that is miscible with water, water, and
non-agglomerated SiO₂ having a particle size of 5-100 nm, are suitable, for example, for the adhesive attachment of direct filling materials, or in combination with an additional neutralizing layer and a fastening cement, for the attachment of indirect, laboratory-produced filling materials.

The invention relates to a light-curing, self-priming, one-component dental adhesive containing nanoparticles.

### BACKGROUND OF THE INVENTION

Adhesion to the dental hard tissue is achieved primarily by mechanical retention to microporous areas and rough surfaces. After cavity preparation, the dental hard tissue must therefore be pretreated before the adhesive is applied in order to obtain an optimally large, retentive, readily wettable adhesive surface. Next, an adhesive with sufficiently low viscosity is necessary that has good wetting characteristics in order to fill the rough surface areas. After the adhesive is processed, the cavity surface is tightly sealed and prepared for accepting the filling material.

Two techniques are widely used for treating the dental hard tissue before the fillings are placed:

1. In the so-called total etching technique, the dental hard tissue is surface-treated by use of phosphoric acid. Both the enamel and dentin are thus etched with phosphoric acid, which is then rinsed off, and excess water is removed by air jet. A characteristic etching pattern is thus obtained on the enamel. The oily layer on the dentin resulting from cavity preparation and which interferes with bonding is removed, and the tubules are exposed. The adhesive component is then applied, the solvent necessary for infiltration is removed by air jet, and the adhesive is radiation-cured.

2. In the use of self-etching adhesives, the steps of acid etching and subsequent application of the adhesive are combined into one step. In the case of the dentin, the acid-containing adhesive systems either dissolve the oily layer and expose the underlying dentin, or merely dissolve the oily layer to make it permeable for the components of the adhesive. Infiltration of monomers into the dental hard tissue takes place at the same time. In the case of the enamel, an etching pattern similar to that from phosphoric acid etching is produced by the acid-containing adhesive systems. Here as well, the solvent necessary for infiltration is removed by air jet, and the adhesive is radiation-cured.

Self-etching adhesives have been known for several years. Such adhesives generally are two-component materials that must either be mixed before use or applied in succession. Separate etching of the tooth surface is not necessary for these materials. The introduction of iBond (Heraeus Kulzer) represents the first self-etching, one-component, single-container adhesive (“all in one adhesive”) that requires no mixing or successive application of the two components. One possible disadvantage of the self-etching, single-container adhesive, however, is that it must be stored under cold conditions (4-10° C.) to avoid degradation during storage. In addition, self-etching, single-container adhesives appear to be sensitive to application errors by the user. This is manifested particularly during the solvent evaporation work step. The adhesive must not be excessively diluted by overly forceful blowing with the air jet, since otherwise the remaining adhesive layer is not thick enough. At the same time, however, as much solvent as possible must be evaporated to ensure complete curing of the adhesive without flaws, and thus to guarantee a secure bond between the dental hard tissue and the filling material. This applies in particular to water, which is necessary for the simultaneous etching and infiltration process but which interferes with reliable final curing of the adhesive.

A dental material is mentioned in EP 803 240 B1 which contains non-agglomerated, nanoparticle SiO₂ fillers in organic dispersants. According to claim 11, these fillers may also be used in adhesive formulations. Marketing information states that the product Xeno® III from Caulk is a self-etching, one-step adhesive containing nanofillers that are a hundred times smaller than conventional hybrid filler particles, but is included in the products that must be mixed immediately before use. CA 2,457,347 A1 relates to a one-component, self-etching, self-priming adhesive, which contains monomers having phosphonic acid groups and which may be combined with nanofillers not specified in greater detail (claim 13). US 20030207960 A1 describes a self-etching, self-priming one-step adhesive based on sulfonic acid, which as filler may contain, among other substances, bound or free silicic acid colloids 10-100 nm in size (paragraph [0021]). US 20030187094 proposes nanoparticles for self-etching, self-priming dental adhesives, each containing an acidic and polymerizable siloxane group. U.S. Pat. No. 6,387,982 B1 proposes polymerizable detergents, preferably in combination with colloidal silicic acids, for use in self-etching, adhesive priming agents.

### SUMMARY OF THE INVENTION

The object of the present invention is to further improve such dental adhesives, particularly with respect to the fillers, in combination with solvents and/or monomers.

The object is achieved by a self-etching, one-component dental adhesive according to claim 1, containing

at least one acrylate or methacrylate monomer,

at least one acidic component,

at least one photoinitiator,

at least one solvent that is miscible with water, water, and

non-agglomerated SiO₂ having a particle size of 5-100 nm.

The presence of multiple crosslinkers and/or thickeners is also preferred.

### DETAILED DESCRIPTION

Considered as thickeners are, on the one hand, polymers such as polyvinylpyrrolidone, polyalkene acids, polyurethanes, polyethylene glycols, starch derivatives, cellulose derivatives, or polymethylmethacrylate having an average molecular weight of around 100,000. Ideally, the thickeners are further modified so that they ultimately contain a polymerizable group, for example polyethylene glycol dimethacrylate.

Pyrogenic silicic acids may be used as further thickening components.

Crosslinkers and multiple crosslinkers are known as such, and contain two or more polymerizable groups per molecule. Alkoxylated pentaerythritol tetraacrylate and/or 1,1,1-trimethylolpropane trimethacrylate and -acrylate in particular may advantageously be used.

The acidic effect in the adhesive is obtained by adding the nanofiller. The dispersion of the nanofillers is surprisingly maintained, contrary to the information in EP 803 240 B1, and the solvent is stable against sedimentation.

The internal bond of the adhesive layer is strengthened by the addition of non-agglomerated SiO₂ nanofiller.

The bond may be further improved by adding a multiple-crosslinking monomer. In addition, improved adherence of the adhesive to the synthetic filling material is also expected.

To reduce the risk of excessive dilution of the adhesive layer, the consistency may be enhanced by use of polymeric thickeners incorporated into the polymer network.

The sensitivity of the adhesive to temperature during storage may be improved, among other measures, by the use of suitable monomers and by modification of the initiator and stabilizer system. This allows storage at room temperature.

The following particular advantages are realized: good film formation properties, high inherent strength of the adhesive, high coefficients of adhesion, and long-term stability during storage despite formulation as a single component.

The adhesive is particularly suited for the attachment of direct filling materials such as composites, compomers, or ormocers, but is also suited for the attachment of indirect, laboratory-produced filling materials in combination with an additional layer, which covers the acidic adhesive component, and a fastening cement. Examples of indirect filling materials are ceramic or composite materials.

Use as fissure sealants may also be considered.

The invention is explained in greater detail by means of the following examples:

### EXAMPLES

1. Dental Adhesive/Mixture of the Following Components:

| | Example | | | | | |
|---|---|---|---|---|---|---|
| | A | B | C | D | E | F |
| Urethane dimethacrylate | 15 | 5 | 5 | 5 | 5 | 5 |
| 4-META | 15 | 15 | 15 | 15 | 15 | 15 |
| Alkoxylated pentaerythrite | | 5 | | | 5 | |
| tetraacrylate | | | | | | |
| Hydroxypropyl methacrylate | | | 5 | | | |
| Aliphatic urethane diacrylate | | | | 5 | | 2 |
| Aerosil 380 | | | | | 2 | 3 |
| Non-agglomerated SiO₂ | | 5 | 5 | 5 | 3 | 5 |
| Acetone | 40 | 40 | 40 | 40 | 40 | 40 |
| Water | 30 | 30 | 30 | 30 | 30 | 30 |
| Camphorquinone | 0.3 | 0.3 | 0.3 | 0.3 | 0.3 | 0.3 |
| 2-n-Butoxyethyl-4- | 0.35 | 0.35 | 0.35 | 0.35 | 0.35 | 0.35 |
| (dimethylamino)benzoate | | | | | | |

(amounts given in grams)

2. Measurement of the Shear Bond:

The effectiveness as acidic monomer in an adhesive formulation was tested by determining the shear bonding strength on dentin and enamel. Human teeth were used that had been kept for a maximum three months after extraction in a 0.5% chloramine-T solution. The teeth were carefully cleaned under running water before being used in the bonding test. On the day before use in the bonding test, the teeth were individually embedded, lying on a proximal side, in cylindrical rubber molds, using Technovit 4001. The teeth were ground, by wet grinding using SiC paper of 80,240, and finally 600 grit, until a sufficiently large dentin or enamel surface was exposed for attaching a plastic cylinder of 2.38 mm diameter. After rinsing with demineralized water, the teeth were dried in an air stream. The preparations from Examples 2A-F were applied by brush to the tooth surface in three layers, dried in a compressed air stream, and irradiated for 20 seconds with the Translux® Energy light unit (Heraeus Kulzer). The samples pretreated in this manner were then clamped beneath a cylindrical plastic mold (2.38 mm diameter, 1 mm height), using a clamping device. Venus® synthetic filling material (Heraeus Kulzer) was then filled into the plastic mold and irradiated for 20 seconds with the Translux® Energy light unit (Heraeus Kulzer). The plastic mold was immediately removed, and the cylindrical sample was stored for 24 hours in warm water at 37° C. until the shear load was introduced. For this purpose, the cylindrical sample was subjected to load in a universal test machine, using a pressure plunger parallel and tight against the ground tooth surface, at a speed of 1 mm/min until the plastic cylinder separated from the tooth. The shear bonding strength is the quotient of the force at rupture and the bonding area, and in each case was determined for 8 samples, for which the average values are given in the table below.

Results:

| | Shear bonding strength |
|---|---|
| Preparation | to dentin (MPa) |
| A | 23.8 |
| B | 31.5 |
| C | 29.1 |
| D | 21.8 |

The adhesive strength of solutions containing non-agglomerated SiO₂ particles was significantly increased.

3. Viscosity Measurements

The viscosity was determined using the Physica UDS 200 rheometer (Paar Physica).

Results:

| Preparation | Viscosity (MPas) |
|---|---|
| A | 2.05 |
| B | 2.84 |
| C | 2.80 |
| D | 4.97 |
| E | 7.48 |
| F | 10.60 |

The consistency was increased by 1.5 to 5 times compared to the unfilled mixtures.

4. Storage Stability Tests

The stability of the mixtures was visually determined. For this purpose, the samples were stored for several days at 50° C. and checked daily for changes. A loss in storage stability was shown by an initial thickening of the solution and/or by sedimentation of the fillers.

Results:

| | Maximum storage at 50° C. |
|---|---|
| Preparation | (days) |
| A | 7 |
| B | 22 |
| C | 8 |
| E | 12 |

After storage at 50° C., Example 2 did not show visual changes in the solution until after 22 days.

1. Light-curing, self-etching one-component dental adhesive comprising
(A) at least one acrylate or methacrylate monomer,
(B) at least one acidic component,
(C) at least one photoinitiator,
(D) at least one solvent that is miscible with water, and
(E) water,
wherein said dental adhesive further comprises
(F) non-agglomerated SiO₂ having a particle size of 5-100 nm.

2. Dental adhesive according to claim 1, further comprising
(G) at least one multiple crosslinker.

3. Dental adhesive according to claim 1, further comprising
(H) at least one thickener.

4. Method for the attachment of indirect, laboratory-produced filling materials in combination with an additional layer, which covers an acidic adhesive component, and a fastening cement, which comprises attaching said materials with the light-curing, self-etching one-component dental adhesive of claim 1.

5. Method according to claim 4, wherein the filling materials are ceramic or composite materials.
